(19) United States
(12) Patent Application Publication
(10) Pub. No.: US 2015/0297728 A1
(43) Pub. Date: Oct. 22, 2015

(54) METHOD OF STABILIZING FOAM IN ALCOHOL-BASED HAND SANITIZERS USING DIMETHICONE POLYOLS AND ALKYLENE OXIDE POLYMERS AS ADJUVENTS

(71) Applicants: Kenneth Charboneau, Paw Paw, MI (US); Peter LeBaron, Paw Paw, MI (US); Thomas Gentle, JR., St. Michael, MN (US)

(72) Inventors: Kenneth Charboneau, Paw Paw, MI (US); Peter LeBaron, Paw Paw, MI (US); Thomas Gentle, JR., St. Michael, MN (US)

(21) Appl. No.: 14/689,659

(22) Filed: Apr. 17, 2015

Related U.S. Application Data
(60) Provisional application No. 61/981,542, filed on Apr. 18, 2014.

(51) Int. Cl. A61K 47/34; A61K 47/10; A61K 31/045

(52) U.S. Cl. CPC A61K 47/34; A61K 31/045; A61K 47/10

(57) ABSTRACT

A method of stabilizing alcohol-based hand sanitizers comprising providing a first composition of matter that is a combination of a linear dimethicone copolyol, an alkylene oxide adjuvant selected from the group consisting of polyethylene oxide, and, polypropylene oxide, and, an alcohol, then, combining the first composition of matter with an alcohol-based hand sanitizer. A composition of matter comprising linear dimethicone copolyol, an alkylene oxide adjuvant selected from the group consisting of polyethylene oxide, and, polypropylene oxide, and, an alcohol, and, a further composition of matter comprising linear dimethicone copolyol, an alkylene oxide adjuvant selected from the group consisting of polyethylene oxide, and, polypropylene oxide, and, an alcohol combined with an alcohol-based hand sanitizer.

This application is a utility application filed from Provisional Patent application Ser. No. 61/981,542, filed Apr. 18, 2014, from which priority is claimed.

### BACKGROUND OF THE DISCLOSURE

This disclosure deals with a method of stabilizing foam in alcohol-based hand sanitizers.

The disclosure relates to a specific structure of silicone glycol surfactant (dimethicone copolyol)that has shown a best-in-class ability to create and sustain foam in a high alcohol-content hand sanitizer formulation when combined with a polyethylene or polypropylene glycol adjuvant.

Solutions containing high alcohol content have demonstrated excellent antiseptic behavior and are commonly used in home and medical hand sanitizers. Alcohol-based hand sanitizers are most commonly gelled with thickeners and water, but these combinations tend to dry out & clog at the delivery tip.

An expanding area of hand sanitization that is also used to deliver high-alcohol antiseptics is to add foam-stabilizing additives and force the water, alcohol & foaming surfactant mixture through an aerosolizing nozzle. This process precludes the need for a pressurized aerosol spray apparatus, which is costly, and relies on the propellant to provide the initial alcohol-containing foam.

The primary shortcoming with an aerosol delivery approach is that when alcohol is the major component, which is required for reasonable antiseptic performance, the low surface tension of the alcohol destabilizes and collapses the generated foam within seconds. Additionally, without a dense foam body from the onset the typical commercial silicone glycol-containing high alcohol solutions have a thin, low viscosity that runs through the fingers and is not consumer friendly.

It is well known that silicone glycols can be used to stabilize the aerosolized alcohol foam. In testing these additives using an aerosol hand pump, however, it was found that results were substandard, with total foam collapse in 10-15 seconds and a highly flowable, runny product using a large range of materials that are covered under the issued patents. Additionally, although the original foam height is much better, in particular, for A-B-A silicone glycols, particularly those linear dimethicone copolyols in the PEG 8 to PEG 12 range, when compared to the pendant silicone glycols, these materials by themselves do not promote the longer term (>90 second) stabilization of the high alcohol foam when compared to the combination of materials described and claimed herein.

High performing formulations of low cost, simple pump-driven aerosols lacking a volatile propellant are made possible with the use of the described silicone glycol in combination with polyethylene glycol or polypropylene glycol.

### THE DISCLOSURE

We have surprisingly discovered that the combination of the linear silicone glycols in combination with polyethylene glycol or polypropylene glycol provides foam retention beyond what is noted when the silicone glycols are used by themselves.

Thus, what is claimed herein is a novel method of stabilizing alcohol-based hand sanitizers comprising providing a first composition of matter that is a combination of a linear dimethicone copolyol, an alkylene oxide adjuvant selected from the group consisting of polyethylene oxide, and, polypropylene oxide, and, an alcohol. Thereafter, combining the first composition of matter with an alcohol-based hand sanitizer.

In a second embodiment, there is a composition of matter comprising a linear dimethicone copolyol; an alkylene oxide adjuvant selected from the group consisting of polyethylene oxide and polypropylene oxide, and an alcohol, and, an alcohol-based hand sanitizer. The alkylene oxide adjuvants are primarily selected from PEG 8 to PEG 12 and polypropylene glycols.

### EXAMPLES

The hand sanitizer formulations were tested using an aerosolizing hand pump (RF-17 Palm Foam machine from Rieke Corp, Auburn, Ind.) with the initial foam height measured after 10 pumps were loaded into a ¾″ diameter glass tube. The initial foam height was recorded (t=0), and the timer was started so that successive foam heights could be measured after 5, 15, 30, 90 and 180 seconds from when the tube was filled with the foam. Each measurement was performed twice and the values were averaged.

The starting fluid for the examples below was comprised of 70% wt. SDA-3C ethanol and 30% purified water. The percent loading and the type of the silicone surfactant is noted in the first column, and the additional foaming adjuvant, if any, is shown in the second column. PEG or PPG refers to polyethylene glycol or polypropylene glycol, respectively, and the number following is the reported molecular weight of the adjuvant.

| ABA silicone | | Foam height | % of t = 0 | % of t = 0 | % of t = 0 | % of t = 0 | % of t = 0 |
|---|---|---|---|---|---|---|---|
| glycol; level | Adjuvant; | (mm), t = 0 | foam after | foam after | foam after | foam after | foam after |
| in 70:30 mix | level | seconds | 5 seconds | 15 seconds | 30 seconds | 90 seconds | 180 seconds |
| PEG 8; 1% | — | 25 | 70% | 52% | 38% | 29% | 3% |
| PEG 10; 1% | — | 28 | 75% | 62% | 40% | 30% | 5% |
| PEG 12; 1% | — | 29 | 78% | 65% | 45% | 35% | 10% |
| PEG 12; 1% | PEG400; 1% | 31 | 99% | 91% | 84% | 71% | 33% |
| PEG 12; 2% | — | 39 | 91% | 70% | 53% | 45% | 15% |
| PEG 12; 2% | PEG400; 2% | 44 | 100% | 100% | 98% | 97% | 93% |
| PEG 12; 2% | PEG400; 5% | 40 | 100% | 100% | 99% | 95% | 90% |
| PEG 10; 2% | PEG400; 2% | 43 | 100% | 100% | 99% | 97% | 92% |
| PEG 10; 2% | — | 38 | 82% | 67% | 48% | 35% | 12% |
| PEG 8; 2% | PEG400; 2% | 43 | 99% | 99% | 95% | 90% | 85% |
| PEG 8; 2% | — | 40 | 85% | 50% | 35% | 20% | 5% |
| PEG 12; 5% | — | 55 | 95% | 82% | 70% | 58% | 40% |
| PEG 12; 5% | PEG400; 2% | 58 | 100% | 98% | 98% | 93% | 92% |
| PEG 12, 2% | PEG100; 2% | 42 | 98% | 98% | 95% | 93% | 89% |
| PEG 12, 2% | PEG1000; 2% | 38 | 98% | 97% | 94% | 94% | 91% |
| PEG 12, 2% | PPG400; 2% | 38 | 99% | 96% | 92% | 92% | 89% |
| PEG 12, 2% | PPG1000; 2% | 35 | 99% | 98% | 91% | 89% | 85% |
| PEG 12, 2% | PEG400; 1%, | 55 | 100% | 100% | 95% | 92% | 84% |
| | PPG400; 1% | | | | | | |

While the initial foam is only moderately impacted by having the foaming adjuvant, the retention of the original foam is greatly impacted as can be seen in the table. Typical use levels of the ABA silicone glycols is in the 1-5% range, but due to their relatively high cost there is a large cost advantage to using less silicone surfactant.

The trends noted here are also evident in different ethanol-to-water ratios, and the same trends exist when other additives were added to the formulation. Generally, the higher the ethanol content and the lower the water (i.e. 75:25 or 80:20), the lower the initial foam height and the faster foam collapse occurs. Similarly, as the ethanol level was dropped and water was increased to a level that is closer to the accepted industry standard as the minimum level required for killing bacteria (60-62% ethanol), then the initial foam height was greater and foam lifetime was longer.

The presence of additives, including additional surfactants, antioxidants, thickeners, fragrances, emollients, dyes, and the like, had a negligible effect on the trends noted above in the data. Only the addition of polyethylene glycol and polypropylene glycol proved to impact foam height, and, more emphatically, foam height retention.

The look of a high density, small bubble size foam is appealing in a hand sanitizer application, however, it is the longevity of the foam that keeps the user manipulating the bacteria-killing solution over the hands.

One hundred percent of bacteria on the hands are not instantly killed the moment that an alcohol solution contacts the skin. The key to high kill rates of disease-causing microbes is extended alcohol contact completely across the hands and under the fingernails. Alcohol that is not frothed into stable foam quickly collapses into a low surface tension fluid that rapidly runs through the user's fingers. Additionally, collapsed foam promotes atmospheric contact with the volatile ethanol solution and results in rapid loss of the product by evaporation. By contrast, stable foam protects the bulk of the ethanol solution from evaporation, keeps the product from running through the fingers, and therefore extends the contact with the hands.

A quick side-by-side comparison of a poor foaming alcohol solution (one that has high initial foam, but >50% collapse after only a few seconds) and a long-lived foam (>90% foam retention after 90 seconds) undeniably demonstrates this effect.

We claim:

1. A novel method of stabilizing alcohol-based hand sanitizers comprising:
I. providing a first composition of matter that is a combination of
i. a linear dimethicone copolyol;
ii. an alkylene oxide adjuvant selected from the group consisting of
a. polyethylene oxide, and,
b. polypropylene oxide, and,
iii. an alcohol;
II. combining said first composition of matter with an alcohol-based hand sanitizer.

2. A composition of matter comprising:
a. a linear dimethicone copolyol;
b. an alkylene oxide adjuvant selected from the group consisting of
i. polyethylene oxide, and,
ii. polypropylene oxide, and,
c. an alcohol.

3. A composition of matter as claimed in claim 2 wherein the alkylene oxide adjuvant is selected from PEG 8 to PEG 12.

4. A composition of matter as claimed in claim 2 wherein the alkylene oxide adjuvant is selected from polypropylene glycol.

5. A composition of matter comprising:
a. a linear dimethicone copolyol;
b. an alkylene oxide adjuvant selected from the group consisting of
i. polyethylene oxide, and,
ii. polypropylene oxide, and,
c. an alcohol;
d. an alcohol-based hand sanitizer.

6. A composition of matter as claimed in claim 5 wherein the alkylene oxide adjuvant is selected from PEG 8 to PEG 12.

7. A composition of matter as claimed in claim 5 wherein the alkylene oxide adjuvant is selected from polypropylene glycol.
